(19) United States
(12) Patent Application Publication
(10) Pub. No.: US 2004/0105839 A1
(43) Pub. Date: Jun. 3, 2004

(54) Biologically active non-antigenic copolymer and conjugates thereof and methods for producing the same

(76) Inventor: Myung-Ok Park, Sungbuk-gu (KR)

Correspondence Address: RODMAN & RODMAN, 7 SOUTH BROADWAY, WHITE PLAINS, NY, 10601, US

(21) Appl. No.: 10/363,874

(22) PCT Filed: Nov. 28, 2002
(86) PCT No.: PCT/KR02/02237

(51) Int. Cl. A61K 31/765; A61K 47/48; C08L 9/02

(52) U.S. Cl. 424/78.17; 525/54.2; 525/233

(57) ABSTRACT

The present invention relates to activated biocompatible non-antigenic copolymers formed by copolymerizing polyethyleneimine with biocompatible polymer other than polyethyleneimine, biologically active non-antigenic conjugates formed by binding said copolymers to biologically active materials such as drugs or proteins. A biologically active non-antigenic conjugate of the present invention has a characteristic feature in that its constitutive copolymer essentially consists of hydrophilic polymer, which plays a role to provide high stability and long in vivo half-life of the hydrophobic drugs or proteins, and positively charged polymer which functions to increase the cellular uptake of the drugs or proteins.

### FIELD OF THE INVENTION

The present invention relates to novel activated biocompatible non-antigenic copolymers which efficiently deliver biologically active materials such as drugs and proteins in vivo through the conjugates made with them. The invention also relates to biologically active non-antigenic conjugates formed by binding activated biocompatible non-antigenic copolymers to biologically active materials. In addition, the invention relates to processes for producing said activated copolymers and conjugates.

### BACKGROUND OF THE INVENTION

A variety of attempts has been made to increase the bioavailability of biologically active materials and/or extend the in vivo half-life of biologically active materials by conjugating them with high-molecular-weight polymers. These polymers have been used solely or as alternative or random copolymers. Typically, polymers or copolymers are activated before they are coupled to biologically active materials.

U.S. Pat. No. 4,179,337 discloses a physiologically active, substantially non-immunogenic water-soluble polypeptide composition comprising a physiologically active polypeptide coupled with a coupling agent to at least one substantially linear polymer having a molecular weight of between about 500 to about 20,000 daltons selected from the group consisting of polyethylene glycol (PEG) and polypropropylene glycol (PPG) wherein the polymer is unsubstituted or substituted by alkoxy or alkyl groups, said alkoxy or alkyl group possessing less than 5 carbon atoms. The polypeptide composition is prepared by reacting terminal carbon atoms bearing a hydroxy group of PEG or PPG with a coupling agent to provide an activated polymer containing a reactive terminal group, and coupling said reactive terminal group of the polymer to a physiologically active immunogenic. PEG or PPG serve to prevent the activity of the polypeptide from being reduced.

Abuchowski, A. and Davis, F. F. reports in Enzymes as Drugs, Holsenberg, J. and Roberts, J., eds. 1981 that PEG can be activated by substituting methylester for one hydroxyl group of PEG and coupling an electrophilic reactive group to another hydroxyl group of PEG. Examples of such activated polymers include PEG-N-hydroxysuccinimide-activated esters bearing an amide bond, PEG-epoxide bearing an alkyl bond, PEG-carbonyl imidazole or PEG-nitrophenyl carbonates bearing a urethane bond, PEG-aldehyde bearing Schiff's base at its N-terminal end, and PEG-hydrazide.

U.S. Pat. No. 5,756,593 describes a method for preparing PEG carboxylic acids in high; purity and water-soluble conjugates formed by coupling the PEG carboxylic acids with drugs such as taxol and camptothecin.

U.S. Pat. No. 5,693,751 claims water-soluble polymerized compounds consisting of a water-soluble block copolymer having a first hydrophilic segment which is a polymer selected from the group consisting of polyethylene glycol, polyacrylamide, polymethacrylamide, polyvinyl pyrrolidone, polyvinyl alcohol, polymethacrylate and polyacrylic ester, and a second hydrophobic segment to a side chain of which a drug is attached, wherein said second segment becomes hydrophobic upon being attached to said drug, said second segment selected from the group consisting of polyaspartic acid, polyglutamic acid, polyacrylic acid, polymethacrylic acid, polymalic acid, polylactic acid and polyalkylene oxide.

However, the foregoing polymer conjugates do not exhibit buffering effect over broad pH range and are incapable of doing efficient cell trafficking and endosomal disruption. As such, they fail to provide the satisfactory efficacy of drug following the entry into cells. Therefore, there is still a need for new polymers to exhibit better buffering effect and enhance the effect of drug or protein in vivo.

### SUMMARY OF THE INVENTION

It was found by the present inventors that the bioavailability of biologically active materials can be maximized by copolymerizing activated biocompatible non-antigenic hydrophilic polymers with positively charged polyethyleneimines (PEIs) to form copolymers, activating the resulting copolymers and binding the activated copolymers to biologically active materials.

Previous reports have disclosed the coupling of PEI to PEG. For examples, The report by Kavanov, A. V., et al. in Bioconjugate Chem. 9 (6), 805-812, 1998 provides a PEG-polycation block copolymer which was synthesized by reacting PEI with PEG activated by 4,4′-dimethoxytrityl (DMT). Prior to its use, the mono-DMT-substituted PEG polymer was purified from the bi-substituted by-products and unreacted initial reagents by performing the prep column chromatography. However, since PEG is a macromolecule, it is difficult to control the number of the bound PEG. It has been reported by Wie, et al. in Int. Archs Allergy Apply. Immun. 64, 84, 1981 that mPEG was converted into the succinyl ester, i.e., mPEG-OCH₂CH₂CONHS, so that it could react with the primary amine of PEI. In addition, the report of R. T. Morrison and R. N. Boyd in Organic Chemistry 735, 740-741, 3rd, 1973 provides the conversion of mPEG into mPEG-aldehyde. However, no mention is made of the function of PEI.

W. T. Godby, et al. state in J. Contr. Rel. 60: 149-160, 1999 that PEI with high molecular weight of 25-800 kDa can be useful for the non-viral delivery of DNA or RNA in vitro or in vivo. Specifically, PEI serves to increase cellular uptake of plasmid DNA via a non-specific adsorption mechanism and exerts the buffering effect within endosomal compartment. As results, PEI prevents degradation of plasmid DNA by enhancing cellular trafficking of plasmid DNA and enables endosomal release of plasmid DNA by lysosomal osmotic swelling and degradation. However, it makes mention of neither the enhancement in the bioavailability of drugs or proteins nor the coupling of PEIs with biocompatible non-antigenic hydrophilic polymers with intention of enhancing the bioavailability of drugs or proteins.

In view of the foregoing, the present invention provides, in one aspect, activated biocompatible non-antigenic copolymers of PEIs and biocompatible polymers other than PEI, capable of binding to biologically active materials and efficiently delivering them in vivo through the conjugate made with them.

In another aspect, the present invention provides processes for producing activated biocompatible non-antigenic copolymers of PEIs and biocompatible polymers other than PEI, which comprises copolymerizing PEIs with activated biocompatible polymers other than PEI to form copolymers and activating the resulting copolymers to produce the said activated copolymers.

In a further aspect, the present invention provides biologically active non-antigenic conjugates capable of efficiently delivering biologically active materials in vivo, wherein said conjugates are formed by binding activated biocompatible non-antigenic copolymers of PEIs and biocompatible polymers other than PEI to said biologically active materials.

In another further aspect, the present invention provides processes for producing biologically active non-antigenic conjugates capable of efficiently delivering biologically active materials in vivo, which comprises copolymerizing PEIs with activated biocompatible polymers other than PEI to form copolymers and, optionally activating the resulting copolymers to form activated copolymers in which the biocompatible polymers bound to PEI are activated, reacting the resulting copolymers with said biologically active materials to produce said conjugates.

In still another aspect, the present invention provides pharmaceutical compositions comprising biologically active non-antigenic conjugates formed by binding activated biocompatible non-antigenic copolymers of PEIs and biocompatible polymers other than PEI to biologically active materials.

### BRIEF DESCRIPTION OF THE DRAWINGS

FIG. 1 is fluorescence microscopy images (100× magnification) showing human hepatoma cellular uptake of PEG, biocompatible non-antigenic copolymer PEG-PEI of the present invention and phosphate-buffered saline (PBS).

FIG. 2 is confocal microscopy images (400× magnification) showing human hepatoma cellular uptake of PEG and biocompatible non-antigenic copolymer PEG-PEI of the present invention.

FIG. 3 shows the uptake level of the conjugate of IFN conjugated with PEI used in the present invention by human hepatoma cells measured by flowcytometry.

FIG. 4 shows the uptake level of the native IFN and the biocompatible non-antigenic conjugate mPEG-PEI-IFN of the present invention by HepG2 cells determined by using radioactive I-125.

### DETAILED DESCRIPTION OF THE INVENTION

An activated non-antigenic biocompatible copolymer of the present invention is represented by the formula I:

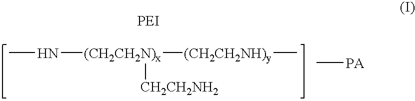

wherein

PEI indicates polyethyleneimine;

x and y are each an integer;

P represents biocompatible non-antigenic polymer; and

A represents reactive functional group or methoxy (CH₃0-).

A biologically active non-antigenic conjugate of the present invention is represented by the formulae IIa, IIb or IIc:

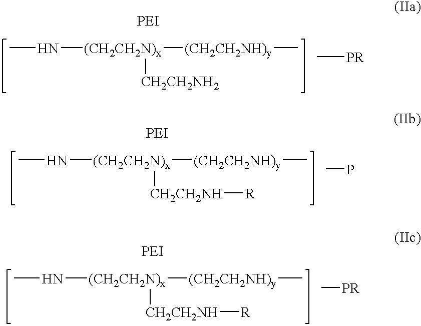

wherein

PEI indicates polyethyleneimine;

x and y are each an integer;

P represents biocompatible non-antigenic polymer; and

R represents biologically active material.

### Polyethyleneimine (PEI)

PEI used to form an activated copolymer of the present invention is a synthetic branched polymer with highly positive charge. It has primary, secondary and tertiary amine groups and thus covers a wide range of pKa, making it furnish a very efficient buffering system. In a preferred embodiment of the present invention, PEI includes but is not limited to pure polyethyleneimine which includes primary, secondary and tertiary amine groups at ratio of about 1:2:1 and has a number average molecular weight of from about 500 daltons to about 20,000 daltons.

In a copolymer of the formula I according to the present invention, biocompatible non-antigenic polymer (P) other than PEI can be covalently bonded to one or both of primary and secondary amine groups existing on PEI. As such, a biologically active material can be directly bonded to either primary amine group or secondary amine group of PEI bonded to other biocompatible non-antigenic polymer (P) and, alternatively, can be bonded to a functional group of biocompatible non-antigenic polymer (P) other than PEI.

### Biocompatible Polymer (P)

A biocompatible polymer bonded to PEI to form an activated copolymer of the present invention is selected from those which can be easily dissolved in various solvents, is substantially non-antigenic and have a number average molecular weight of from about 200 daltons to about 25,000 daltons. A preferred biocompatible polymer includes but is not limited to polyethylene glycol (PEG), polypropylene glycol (PPG), polyoxyethylene (POE), polytrimethylene glycol, polylactic acid and derivatives thereof, polyacrylic acid and derivatives thereof, polyamino acid, polyurethane, polyphosphazene, polyalkylene oxide (PAO), polysaccharide, dextran, polyvinyl pyrrolidone, polyvinyl alcohol (PVA), polyacryl amide and similar non-antigenic polymers. In addition, copolymers consisting of at least two polymers as exemplified above can be used as a biocompatible polymer (P) according to the present invention.

In a preferred embodiment of the present invention, polyalkylene oxide is represented by the formula:

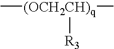

wherein q is an integer of from 10 to 600 and R₃ is a hydrogen or C₁₋₅ alkyl.

In another embodiment of the present invention, biocompatible polymer (P) is a branched polymer which can lead to second and third branching from the biologically active material. In addition, bifunctional and hetero-bifunctional activated polymer esters can be used as the biocompatible polymer according to the present invention. The polymer (P) used in the present invention can also be copolymerized with a bifunctional material, for example poly(alkylene glycol) diamine, to form a useful interpermeable network for permeable contact lenses, wound dressing, drug delivery system, etc.

### Reactive Functional Group (A)

In an activated copolymer of the formula I according to the present invention, “A” can be a reactive functional group. The term “reactive functional group” indicates an activating group or moiety for a biocompatible polymer (P) which is capable of binding to a biologically active material. One or more terminal groups of the biocompatible polymer can be converted into functionalized reactive group so that it can undergo binding to a biologically active material. Such a process is called “activation”. The product resulting from the process is “activated biocompatible copolymer”. For example, in order to conjugate poly(alkylene oxide) with a biologically active material, one of terminal groups of the polymer can be converted into a reactive functional group such as carbonate. The product obtained thereby is an activated poly(alkylene oxide).

The reactive functional group (A) of the formula I can be selected from the group consisting of (i) functional groups capable of reacting with an amino group, for example, (a) carbonates such as p-nitrophenyl and succinimidyl, (b) carbonyl imidazole, (c) azlactones, (d) cyclic imide thiones or (e) isocyanates or isothiocyanates; (ii) functional groups capable of reacting with carboxylic acid groups and reactive carbonyl groups, for example, (a) primary amines or (b) hydrazine and hydrazide functional groups such as acyl hydrazides, carbazates, semicarbamates and thiocarbazates; (iii) functional groups capable of reacting with mercapto or sulfhydryl groups, for example, phenyl glyoxals; (iv) functional groups capable of reacting with hydroxyl groups, for example, carboxylic acid; and (v) other nucleophiles capable of reacting with an electrophilic center.

A preferred reactive functional group (A) of the present invention includes but is not limited to N-hydroxysuccinimide ester (NHS), hydrazine hydrate (NH₂NH₂), carbonyl imidazole, nitrophenyl, isocyanate, sulfonyl chloride, aldehyde, glyoxal, epoxide, carbonate, cyanuric halide, dithiocarbonate, tosylate and maleimide.

### Preferred Embodiment of Activated Copolymer

In one preferred embodiment of the present invention, a biocompatible copolymer includes one represented by the formula Ia:

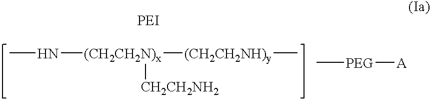

wherein x, y and A are the same as defined above.

A preferred copolymer of the formula Ia includes, but is not limited to, one represented by the formulae:

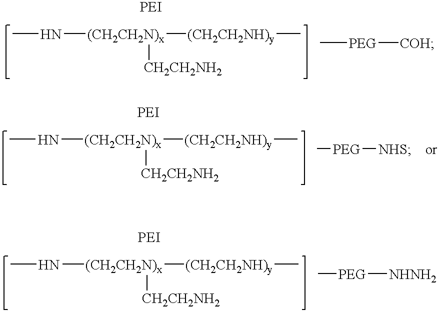

wherein x and y are the same as defined above.

Another preferred embodiment of the present invention provides copolymers containing a terminal carboxylic acid group which is useful in the formation of ester-based prodrugs. The copolymers are of the formula Ib:

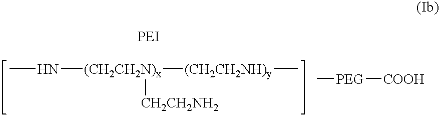

wherein x and y are the same as defined above.

### Preparation of Activated Biocompatible Copolymer of Formula I

A process for producing an activated biocompatible non-antigenic copolymer of formula I comprises the steps of (a) activating a biocompatible polymer (P) and reacting the resulting activated biocompatible polymer with PEI to form a copolymer PEI-P, (b) activating the resulting copolymer PEI-P to produce said activated biocompatible non-antigenic copolymer.

One method for activating polymer (P) includes first functionalizing with compounds capable of activating the hydroxyl group such as p-nitrophenyl chloroformate to form a reactive p-nitrophenyl carbonate. The resulting p-nitrophenyl carbonate polymer can be directly reacted with a biologically active material. The p-nitrophenyl carbonate polymer can also serve as an intermediate. It can be reacted with a large excess of N-hydroxysuccinimide to form a succinimidyl carbonate-activated branched polymer. Alternatively, a p-nitrophenyl carbonate polymer intermediate can be reacted with anhydrous hydrazine to form a carbazates branched polymer.

Polymer can also be activated by reacting with an alkyl haloacetate in the presence of base to form an intermediate alkyl ester of the corresponding polymeric carboxylic acid and thereafter reacting the intermediate alkyl ester with an acid such as trifluoroacetic acid to form the corresponding polymeric compound containing a terminal carboxylic acid. In carrying out the reaction, the molar ratio of the alkyl haloacetate to the polymer is greater than 1:1. The second step for reacting alkyl ester with acid is carried out at a temperature of from about 0° C. to about 50° C., and preferably at a temperature of from about 20° C. to about 30° C. Optionally, the second step can be carried out in the presence of water. Preferably, tertiary alkyl haloacetates of the formula:

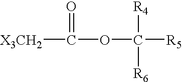

wherein X₃ is chlorine, bromine or iodine; and R₄, R₅ and R₆ are independently selected from the group consisting of C₁₋₈ alkyl, C₁₋₈ substituted alkyl or C₁₋₈ branched alkyl and aryl. Preferred tertiary alkyl haloacetates include tertiary butyl haloacetates such as t-butyl bromoacetate or t-butyl chloroacetate. Suitable bases include potassium t-butoxide or butyl lithium, sodium amide and sodium hydride. Suitable acids include trifluoroacetic acid or sulfuric, phosphoric and hydrochloric acid.

Polymers having a terminal functional amino group can be activated by reacting with hydroxyl acid, for example, lactic acid and glycolic acid, to form hydroxy amide and functionalizing the hydroxy amide with p-nitrophenyl chloroformate.

### Biologically Active Material (R)

In another aspect, the present invention provides biologically active non-antigenic conjugates formed by binding biologically active materials to activated biocompatible copolymers of the formula I.

The term “biologically active material” indicates drugs or proteins which covalently bind to activated biocompatible copolymers of the present invention to form conjugates in which at least portion of inherent physiological or pharmacological activity of the drugs or proteins remains. The biologically active material of the present invention includes all of chemically synthesized or naturally isolated drugs and proteins.

Examples of the biologically active materials of the present invention are drug, preferably hydrophobic drug, enzyme, hormone, polypeptide, peptide, biologically active small molecules, cytokine and anticancer drug.

Polypeptides and peptides of interest include, but are not limited to, hemoglobin, serum proteins (for example, blood factors including Factors VII, VIII, and IX), immunoglobulins, cytokines (for example, interleukins), alpha-, beta- and gamma-interferons, colony stimulating factors including granulocyte colony stimulating factors, platelet derived growth factors (PDGF) and phospholipase-activating protein (PLAP). Other proteins of general biological or therapeutic interest include insulin, plant proteins (for example, lectins and ricins), tumor necrosis factors (TNF) and related alleles, growth factors (for example, tissue growth factors and epidermal growth factors), hormones (for example, follicle-stimulating hormone, thyroid-stimulating hormone, antidiuretic hormones, pigmentary hormones, PARATHYROID and progesterone-releasing hormone and derivatives thereof), calcitonin, calcitonin gene related peptide (CGRP), synthetic enkephalin, somatomedins, erythropoietin, hypothalamic releasing factors, prolactin, chorionic gonadotropin, tissue plasminogen activator, growth hormone releasing peptide (GHRP), thymic humoral factor (THF) and the like. Immunoglobulins of interest include IgG, IgE, IgM, IgA, IgD and fragments thereof.

The present invention is particularly suitable for poorly soluble drugs which have few or even a single attachment site for copolymer conjugation such as medicinal chemicals whether isolated from nature or synthesized. Examples of pharmaceutical chemicals are anti-tumor agents such as paclitaxel, Taxotere and analogs thereof, taxoid molecules, camptothecin, anthracyclines and methotrexates, cardiovascular agents, gastrointestinal agents, central nervous system-activating agents, analgesics, fertility or contraceptive agents, anti-inflammatory agents, steroidal agents, cardiovascular agents, vasodilating agents, vasoconstricting agents and the like.

The biologically active materials of the present invention also include any portion of a polypeptide demonstrating in vivo bioactivity. This includes amino acid sequences, antibody fragments, binding molecules including fusions of antibodies or fragments, polyclonal antibodies, monoclonal antibodies, catalytic antibodies and the like. Other proteins of interest are allergen proteins such as ragweed, Antigen E, honeybee venom, mite allergen, and the like.

Enzymes of interest include carbohydrate-specific enzymes, proteolytic enzymes, oxidoreductases, transferases, hydrolases, lyases, isomerases and ligases. Without being limited to particular enzymes, examples of enzymes of interest include asparaginase, arginase, arginine deaminase, adenosine deaminase, superoxide dismutase, endotoxinases, catalases, chymotrypsin, lipases, uricases, adenosine diphosphatase, tyrosinases and bilirubin oxidase. Carbohydrate-specific enzymes of interest include glucose oxidases, glucosidases, galactosidases, glucocerebrosidases, glucouronidases, etc.

### Biologically Active Non-Antigenic Conjugate and Preparation Thereof

In one embodiment of the present invention, there is provided a biologically active non-antigenic conjugate of formula IIa:

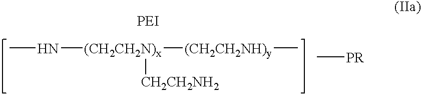

wherein PEI, x, y, P and R are the same as defined above. According to the compounds of the formula IIa, the biologically active material is bonded, via the biocompatible polymer, to one or both of primary and secondary amines of PEI.

Another embodiment of the present invention provides a biologically active non-antigenic conjugate of the formula IIb:

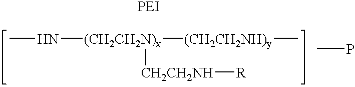

wherein PEI, x, y, P and R are the same as defined above. According to the compound of formula IIb, biologically active material and biocompatible polymer are bonded to primary and secondary amines of PEI, respectively.

In still another embodiment of the present invention, there is provided a biologically active non-antigenic conjugate of the formula IIc:

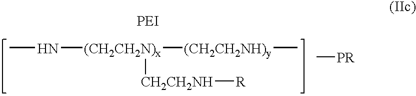

wherein PEI, x, y, P and R are the same as defined above. According to the compounds of the formula IIc, one biologically active material is bonded to primary amine of PEI and another biologically active material is bonded, via the biocompatible polymer, to secondary amine of PEI.

A process for producing biologically active non-antigenic conjugates comprises contacting activated biocompatible copolymers with biologically active materials under the sufficient conditions to conjugate them while maintaining at least portion of inherent activity of the biologically active material. Alternatively, biologically active non-antigenic conjugates can be prepared by reacting activated biocompatible polymers with biologically active materials to form conjugates and then reacting the resulting conjugates with PEIs to produce the desired conjugates.

A stoichiometric excess of activated copolymer is reacted with biologically active materials to produce the conjugates. For example, peptide-copolymer, enzyme-copolymer, antibody-copolymer and drug-copolymer conjugates are prepared by reacting biologically active materials with activated biocompatible copolymers at the ratio of from about 1:1 to about 1:100, preferably at the ratio of from 1:I to 1:20.

Biologically active materials can be reacted with activated biocompatible copolymers in an aqueous reaction medium which can be buffered, depending upon the pH requirements of the biologically active material. The optimum pH for the reaction is generally between about 6.5 and about 8.0 and preferably about 7.4 for proteinaceous/polypeptide materials. Organic/chemotherapeutic moieties can be reacted in non-aqueous systems. The optimum reaction condition for the biologically active material's stability, reaction efficiency, etc. is within level of ordinary skill in the art. The preferred temperature range is between 4° C. and 37° C. The temperature of the reaction medium cannot exceed the temperature at which the biologically active material may denature or decompose. It is preferred that biologically active materials be reacted with an excess of activated copolymers for from five minutes to 10 hours. Following the reaction, the conjugates are recovered and purified such as by column chromatography, diafiltration, combinations thereof, or the like.

### Preferred Embodiment of Biologically Active Non-Antigenic Conjugate

In a preferred embodiment of the present invention, the biologically active non-antigenic conjugates are represented by the formulae:

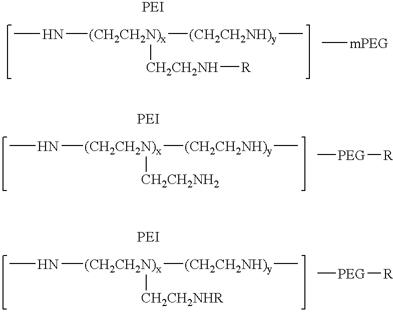

wherein mPEG indicates methoxypolyethylene glycol and R represents biologically active material.

As one example to produce the biologically active non-antigenic conjugates of the present invention, the mPEG-PEI-drug conjugate can be obtained by reacting PEI with mPEG-OCH₂CH₂CONHS to form mPEG-PEI copolymer and thereafter reacting the resulting mPEG-PEI copolymer with drug. As another example, mPEG-PEI-protein can be obtained by reacting PEI with mPEG-CHO to form mPEG-PEI copolymer and thereafter reacting the resulting mPEG-PEI copolymer with drug. As a further example, PEI-PEG-drug conjugate can be obtained by reacting activated polymers NH₂-PEG-OCH₂CH₂CONHS with drugs to form the conjugates PEG-drug and thereafter reacting the resulting conjugates with PEIs.

### Pharmaceutical Composition

In another aspect of the present invention, there is provided a method for the treatment of various medical conditions in mammals, preferably, humans which comprises administering a biologically active non-antigenic conjugate to said subject. The biologically active materials for the biologically active non-antigenic conjugates can be selected properly according to the medical conditions to be treated. For example, where interferon is used as the biologically active material, the medical conditions to be treated by using it include, but are not limited to, cell proliferative disease, especially cancer (for example, Kaposi's sarcoma, ovarian cancer and multiple myeloma) and virus infection (for example, herpes simplex, cytomegalovirus and Epstein-Barr virus).

The dosage of the biologically active materials varies depending on the types of the biologically active materials, patient's condition and severity, etc. as well known in the art. The proteins are generally administered once per two days and preferably once to three times a week. For example, the interferon protein is administrated in an amount of about 5×10⁶ units 3 times a week by intravenous injection. However, doses of the biologically active materials to be administered as the conjugate forms of the present invention can be lowered by from about 20% to about 80% of the usually available doses.

The biologically active non-antigenic conjugates of the present invention can be formulated in combination of pharmaceutically acceptable carriers. The pharmaceutical formulations can be prepared by routine methods. Examples of the carriers are adjuvants such as Tris-HCl and acetate or phosphate buffer solutions, carriers such as human serum albumin, diluents such as polyoxyethylene sorbitan, preservatives such as thimerosol and benzyl alcohol, solubilizers, etc. The pharmaceutical composition containing the conjugates of the present invention can be in forms of solution, suspension, tablet, capsule, lyophilized and dry powder as readily prepared by well known methods in the art. The formulations can be administered intravenously, subcutaneously, intramuscularly, orally, nasally and through other allowable systemic or local routes.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

### Example 1

### Preparation of mPEG-PEI Copolymer

1 g of mPEG(MW5,000)-NHS (N-hydroxysuccinimidyl) (0.2 mmole) and 0.4 g of PEI (MW2000) (Sigma-Aldrich) (0.2 mmole) were dissolved in 100 ml of acetonitrile at room temperature for 48 hours. After completion of the reaction, the resulting solution was extracted three times with methylene chloride. The fractions were dried over Na₂SO₄, filtered and evaporated. The remaining product was crystallized from isopropyl alcohol in a cold bath to yield a white solid which was filtered, washed with ether and dried under vacuum to afford 1 g of the title copolymer, which had a molecular weight of about MW7,000 daltons, as a white solid.

### Example 2

### Preparation of mPEG-PEI-FITC

10 mg of mPEG-PEI obtained from Example 1 was dissolved in 1 ml of 0.1 N sodium bicarbonate, pH 8.5. The resulting solution was added to a buffer solution of 1 mg of fluorescein isothiocyanate (FITC) in 200 μl of dimethyl sulfoxide (DMSO). The reaction solution was kept at room temperature for about 2 hours. Excess of FITC was then removed using Bio-Gel P-10 column (Bio-Rad Laboratories) to yield mPEG-PEI-FITC which was stored in portions at −20° C.

### Example 3

### Preparation of PEG-FITC

5 mg of PEG diamine (2 KDa) was dissolved in 0.5 ml of 0.1 N sodium bicarbonate solution, pH 8.2. The resulting solution was added to a buffer solution of 0.5 mg of FITC in 100 μl of DMSO. The reaction solution was kept at room temperature for about 2 hours. Excess of FITC was then removed using Bio-Gel P-column (Bio-Rad Laboratories) to yield mPEG-PEI-FITC which was stored in portions at −20° C.

### Example 4

### Preparation of PEI-IFN-FITC

2.2 mg of PEI (Sigma-Aldrich) and 2 mg of 1-(3-dimethylaminopropyl-3-ethylcarbodiimide (EDAC) were added to a solution of 2 mg of interferon α-2a (IFN) in 0.1 N sodium bicarbonate-buffered solution, pH 7 which was in turn exchanged to 0.1 N sodium bicarbonate-buffered solution having the pH of 8. The copolymer IFN-PEI obtained thereby was mixed with 2 equivalents of FITC. The mixture was reacted at room temperature for 1 hour and then excess of FITC was removed using Bio-Gel P-10 column to yield PEI-IFN-FITC.

### Example 5

### Preparation of mPEG-PEI-IFN

3.3 mg of mPEG-PEI copolymer prepared by Example 1 was added to a solution of 2 mg of IFN in 0.1 N sodium phosphate buffer solution, pH 7, followed by addition of 1 mg of EDAC. The reaction solution was kept at room temperature for 2 hours and then at 4° C. for 12 hours to afford mPEG-PEI-IFN.

### Example 6

### Preparation of Activated Aldehyde-PEG-PEI

1 g of aldehyde-PEG (Shearwater) (MW5,000, 0.2 mmole) and 0.4 g of PEI (MW2,000, 0.2 mmole) were dissolved in 100 ml of acetonitrile at room temperature for 48 hours. The reaction solution was extracted three times with methylene chloride. The fractions were dried over Na₂SO₄, filtered and evaporated. The remaining product was crystallized from isopropyl alcohol in a cold bath to yield a white solid which was filtered, washed with ether and dried in vacuo to afford 0.8 g of the title copolymer.

### Example 7

### Preparation of Conjugate of PEG-PEI Copolymer with Paclitaxel

56 mg of paclitaxel (0.07 mmole) and 100 μl of nitrophenyl chloroformate (0.14 mmole) were reacted with 10 ml of acetonitrile at room temperature for 2 hours. To the reaction solution 100 mg of PEG-PEI (0.014 mmole) prepared by Example 6 was added. The resulting mixture was kept at 25° C. for 12 hours. The reaction product was crystallized from isopropyl alcohol in a cold bath to yield a white solid which was filtered, washed with ether and dried in vacuo to afford 120 mg of the conjugate PEG-PEI-paclitaxel as a white solid.

### Example 8

### Preparation of Conjugate of mPEG-PEI Copolymer with Paclitaxel

110 mg of paclitaxel (0.14 mmole) and 200 μl of nitrophenyl chloroformate (0.28 mmole) were reacted with 20 ml of acetonitrile at room temperature for 2 hours. To the reaction solution 100 mg of mPEG-PEI (0.014 mmole) prepared by Example 1 was added. The resulting mixture was kept at 25° C. for 12 hours. The reaction product was crystallized from isopropyl alcohol in a cold bath to yield a white solid which was filtered, washed with ether and dried in vacuo to afford 180 mg of the conjugate PEG-PEI-paclitaxel as a white solid.

### Example 9

### Preparation of Activated PEG Having a Heterofunctional Terminal Group (NH₂-PEG-OCH₂CH₂CONHS)

3 g of NH₂PEG-OCH₂COOH (MW5,000) (0.6 mmole) (prepared by Sepulchre, M. et al. in Makromol. Chem. 184, 1849-1859, 1983) was dissolved in methylene chloride. To the solution 0.2 g of N-hydroxysuccinimidyl(NHS) (1.8 mmole) and 0.3 g of N,N′-dicyclohexyl carbodiimide (1.8 mmole) were added. The reaction mixture was stirred at 30° C. for 24 hours. After completion of the reaction, the solution was cooled to room temperature. The solution was filtered through Celite and coal in sequence, and then evaporated. The remaining product was crystallized from isopropyl alcohol in a cold bath to yield a white solid which was filtered, washed with ether and dried in vacuo to afford 2.81 g (yield 91%) of the title compound NH₂-PEG-OCH₂CH₂CONHS (MW5,000) as a white solid.

### Example 10

### Preparation of Conjugate (Oaclitaxel-NH-PEG-OCH₂CH₂CONHS) of Activated PEG Having a Heterofunctional Terminal Group with Paclitaxel

56 mg of paclitaxel (0.07 mmole) and 10 ml of nitrophenyl chloroformate (0.14 mmole) were reacted with 20 ml of acetonitrile at room temperature for 2 hours. To the reaction solution 70 mg of NH₂-PEG-OCH₂CH₂CONHS (0.014 mmole) prepared by Example 9 was added. The resulting mixture was kept at 25° C. for 12 hours. The solvent was removed by rotary evaporation. The remaining product was crystallized from isopropyl alcohol in a cold bath to yield a white solid which was filtered, washed with ether and dried in vacuo to afford 85 mg of the title conjugate as a white solid.

### Example 11

### Preparation of Conjugate PEI-PEG (MW5,000)-Paclitaxel

50 mg of PEG (MW5,000)-paclitaxel (0.008 mmole) prepared by Example 11 and 20 mg of PEI (MW2,000) (Sigma-Aldrich) (0.008 mmole) were reacted with 100 ml of acetonitrile at 25° C. for 48 hours. The reaction mixture was extracted three times with methylene chloride. The fractions were dried over Na₂SO₄, filtered and evaporated. The remaining product was crystallized from isopropyl alcohol in a cold bath to yield a white solid which was filtered, washed with ether and dried in vacuo to afford 60 mg of the title conjugate as a white solid.

### Experimental Example 1

### Fluorescence Microscopy Analysis

Human liver carcinoma HepG2 cells were seeded into 8-well chamber slide at a density of 2×10⁴ cells/well. 200 μl of minimum essential media (MEM) was put into the chamber slide and cultured for 24 hours at 37° C. under 5% CO₂. The seed cells in each well were fixed with 70% EtOH at −20° C. for 20 minutes and blocked with 1% BSA/PBS at room temperature for 15 minutes. PBS (control), PEG-FITC sample prepared by Example 3, and mPEG-PEI-FITC sample prepared by Example 2 were added to each well and the slide was cultured at 37° C. for 1 hour. After the slide was mounted with antibleaching solution, it was observed using a fluorescence microscope (100× magnification).

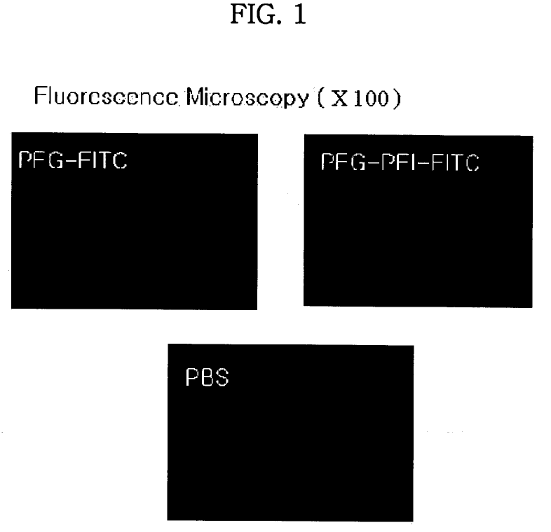

The fluorescence microscope images are shown in FIG. 1. The image of PBS is seen black, indicating that no PBS was absorbed into HepG2 cells. The image of PEG reveals so very low fluorescence, indicating that PEG was little uptaken by HepG2 cells. As contrast, the PEG-PEI copolymer of the present invention resulted in high fluorescence. It is evident from the result that the high uptake of the PEG-PEI copolymer by HepG2 cells was achieved.

### Experimental Example 2

### Confocal Microscopy Analysis

Human liver carcinoma HepG2 cells were seeded into 8-well chamber slide at a density of 2×10⁴ cells/well. 200 μl of MEM was put into the chamber slide and cultured for 24 hours at 37° C. under 5% CO₂. The seed cells in each well were fixed with 2% formaldehyde at room temperature for 20 minutes and blocked with 1% BSA/PBS at room temperature for 15 minutes. PEG-FITC sample prepared by Example 3 and mPEG-PEI-FITC sample prepared by Example 2 were added to each well and the slide was cultured at 37° C. for 1 hour. After the slide was mounted with antibleaching solution, it was observed using a fluorescence microscope (400× magnification).

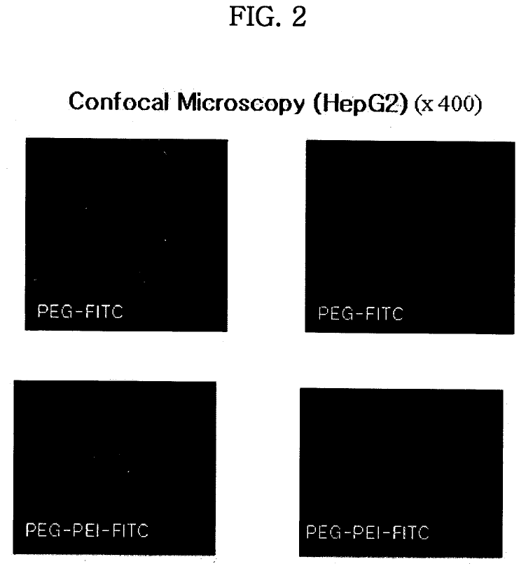

The fluorescence microscope images are shown in FIG. 2. It can be seen from the images that PEG was uptaken by HepG2 cells but was conglomerated around the nucleus of HepG2 cells, demonstrating that the uptake of PEG into the nucleus of HepG2 cells was not substantially made. As contrast, the PEG-PEI copolymer of the present invention was uptaken into the nucleus of HepG2 cells.

### Experimental Example 3

### Flow Cytometry

Human liver carcinoma HepG2 cells were put in E-tube at a density of about 2×10⁴ cells/well. To the E-tube IFN-PEI-FITC sample prepared by Example 4 and native IFN were added at various concentrations. The reaction was allowed at 37° C. for 1 hour. The reaction mixture was centrifuged at 12,000 g for 30 seconds to remove excess of FITC sample. FITC-bound cells were fixed by 200 μl of 1% formaldehyde at 4° C. for 15 minutes. The uptake of FITC sample by cells was measured by flow cytometer.

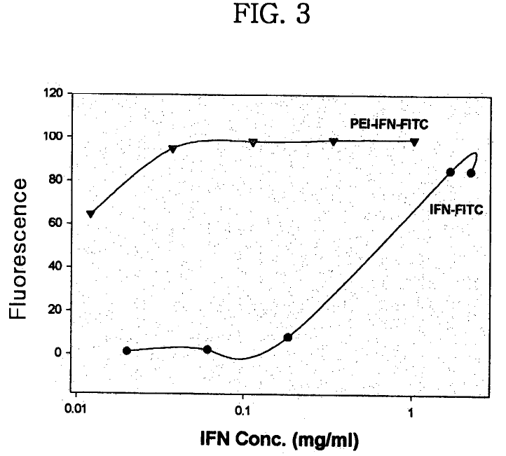

The results are shown in FIG. 3. It can be seen from FIG. 3 that high amounts of the conjugate PEI-IFN was uptaken by human liver carcinoma HepG2 cells.

### Experimental Example 4

### Cellular Uptake Experiment Using I-125

About 400-500 μg of IFN or mPEG-PEI-IFN prepared by Example 5 was dissolved in PBS at the final concentration of 2-3 mg/ml. The resulting solution was added to two IODO-BEADS (Pierce Chemical Company) which was previously reacted in [I-125]NaI for 5 minutes. The reaction was allowed for 10 minutes. The unreacted NaI was removed by running P-10 column. The concentration of I-125 in each sample was measured by Gamma Counter (Beckman Coulter, Inc.). Each sample was stored at 4° C.

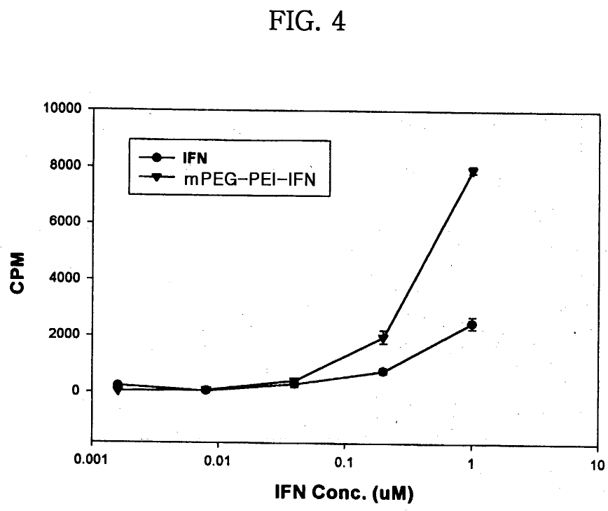

HepG2 cells were seeded into 24-well chamber slide at a density of 2×10⁵ cells/well. [I-125]-labeled sample was added to each well at various concentrations. The reaction was allowed at 37° C. for 1 hour. The wells were washed with PBS. After the cells were suspended in 1 N NaOH, the amount of I-125 was measured by Gamma Counter (Beckman Coulter, Inc.). The results are shown in FIG. 4. It is generally known that the uptake of PEG-grafted IFN by cells is lower than that of native IFN. In this regard, it is evident from FIG. 4 that PEI considerably increases the uptake of IFN by cells.

A biologically active non-antigenic conjugate of the present invention has a characteristic feature in that its constitutive copolymer essentially consists of hydrophilic polymer and positively charged polymer. While the hydrophilic polymer playing a role to provide high stability and long in vivo half-life of the hydrophobic drugs or proteins, the positively charged polymer functions to increase the cellular uptake of the drugs or proteins.

What is claimed is:

1. An activated biocompatible non-antigenic copolymer of the formula I: 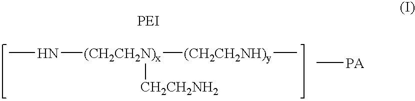
wherein PEI indicates polyethyleneimine; x and y are each an integer; P represents biocompatible non-antigenic polymer; and A represents reactive functional group or methoxy (CH₃O—).

2. The copolymer of claim 1 in which said biocompatible non-antigenic polymer is selected from the group consisting of polyethylene glycol, polypropylene glycol, polyoxyethylene, polytrimethylene glycol, polylactic acid and derivatives thereof, polyacrylic acid and derivatives thereof, polyamino acid, polyurethane, polyphosphazene, polyalkylene oxide, polysaccharide, dextran, polyvinyl pyrrolidone, polyvinyl alcohol, polyacryl amide and similar non-antigenic polymers.

3. The copolymer of claim 1 in which said PEI includes pure polyethyleneimine having primary, secondary and tertiary amine groups at the ratio of about 1:2:1 and having a number average molecular weight of from about 500 to about 20,000.

4. The copolymer of claim 2 in which said polyalkylene oxide includes polyethylene glycol represented by the formula: 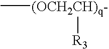
wherein q is an integer of from 10 to 600; and R₃ is a hydrogen or C₁₋₅ alkyl.

5. A process for producing an activated biocompatible non-antigenic copolymer of the formula I: 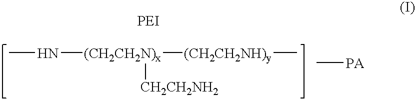
wherein PEI indicates polyethyleneimine; x and y are each an integer; P represents biocompatible non-antigenic polymer; and A represents functional group or methoxy (CH₃O—), which comprises (a) activating a biocompatible polymer (P) and reacting the resulting activated biocompatible polymer with PEI to form copolymer PEI-P, (b) activating the resulting copolymer PEI-P to produce said activated biocompatible non-antigenic copolymer.

6. A biologically active non-antigenic conjugate of the formulae (IIa), (IIb) or (IIc): 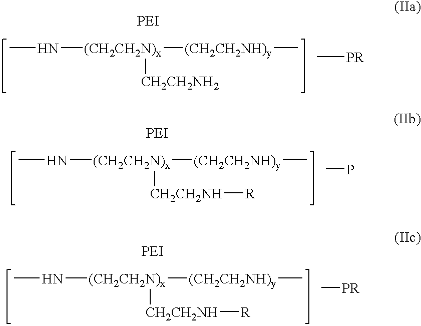
wherein PEI indicates polyethyleneimine; x and y are each an integer; P represents biocompatible non-antigenic polymer; and R represents biologically active material.

7. The conjugate of claim 6 in which said biologically active material is selected from the group consisting of adriamycin, daunomycin, paclitaxel, methotrexate, mitomycin C, drugs involved in central nervous system or peripheral nervous system, antiallergic drug, respiratory system drug, hormonal drug and antibiotics.

8. The conjugate of claim 6 in which said biologically active material is selected from the group consisting of alpha-, beta and gamma-interferon, asparaginase, arginase, arginin diiminase, adenosine deaminase, superoxide dismutase, endotoxinase, catalase, kimotrypsine, lipase, urikase, adenosine diphosphotase, tyrosinase, glucose oxidase, glucosidase, galactosidase, glucouronidase, hemoglobin, blood factor VII, VIII and IX, immunoglobuline, interleukine, G-CSF, GM-CSF, PDGF, lectin, lysin, TNF, TGFs, EGF, PTH, calcitonin, parathyroid hormone, insulin, synthetic enkephalin, growth hormone-releasing factor peptide, progesterone-releasing hormone and derivatives thereof, hypothalamic releasing factors, calcitonin gene-related peptide, thyrotropin-stimulating hormone and thymus humoral factor.

9. The conjugate of claim 6 in which said biocompatible non-antigenic polymer (P) is selected from the group consisting of polyethylene glycol, polypropylene glycol, polyoxyethylene, polytrimethylene glycol, polylactic acid and derivatives thereof, polyacrylic acid and derivatives thereof, polyamino acid, polyurethane, polyphosphazene, polyalkylene oxide, polysaccharide, dextran, polyvinyl pyrrolidone, polyvinyl alcohol and polyacryl amide.

10. The conjugate of claim 6 in which said PEI includes pure polyethyleneimine having primary, secondary and tertiary amine groups at the ratio of about 1:2:1 and having a number average molecular weight of from about 500 to about 20,000.

11. The conjugate of claim 9 in which said polyalkylene oxide includes polyethylene glycol represented by the following formula: 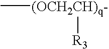
wherein q is an integer of from 10 to 600; and R₃ is a hydrogen or C₁₋₅ alkyl.

12. A pharmaceutical composition comprising a biologically active non-antigenic conjugate of claim 6 and a pharmaceutically acceptable carrier.
